(19) United States
(12) Patent Application Publication
(10) Pub. No.: US 2025/0143984 A1
(43) Pub. Date: May 8, 2025

(54) Herbal Oral Care Compositions

(71) Applicant: Colgate-Palmolive Company, New York, NY (US)

(72) Inventors: Manish MANDHARE, Mumbai (IN); Nikhil Vamanrao MALI, Kaylan (IN); Purushottam Dagadu JADHAV, Maharashtra (IN); Neelima Jitendra UTGIKAR, Dombivli (IN); Bhagyashri CHAVAN, Mumbai (IN); Fusong SUN, Hillsborough, NJ (US); Edna AMBUNDO, Cranbury, NJ (US)

(73) Assignee: Colgate-Palmolive Company, New York, NY (US)

(21) Appl. No.: 18/835,957

(22) PCT Filed: Feb. 3, 2023
(86) PCT No.: PCT/US2023/012315

§ 371 Date: Aug. 5, 2024

(30) Foreign Application Priority Data
Feb. 5, 2022 (IN) 202211006252

(51) Int. Cl. A61K 8/27; A61K 8/19; A61K 8/25; A61K 8/73; A61K 8/9789; A61Q 11/00

(52) U.S. Cl. CPC A61K 8/27; A61K 8/19; A61K 8/25; A61K 8/73; A61K 8/731; A61K 8/9789; A61Q 11/00; A61K 2800/28; A61K 2800/48

(57) ABSTRACT

Described herein are oral care compositions comprising a first source of zinc comprising zinc oxide; a second source of zinc comprising zinc citrate; a gum system comprising xanthan gum and carboxymethyl cellulose; a carrier comprising calcium carbonate (e.g., natural calcium carbonate and precipitated calcium carbonate); a natural ingredient component comprising at least one herbal extract(s) and at least one essential oil; and at least one oleoresin (e.g., ginger and/or pepper). The disclosure also contemplates methods of using the formulations described herein.

### CROSS REFERENCE TO RELATED APPLICATIONS

This application claims priority benefit of India application Ser. No. 202211006252 filed Feb. 5, 2022, the entire contents of which are incorporated herein.

### BACKGROUND

There is an interest in providing oral care products which have a natural flavor profile. In particular, oral care products containing a mixture of herbal ingredients, such as those reported in Ayurvedic texts.

However, the degree of antibacterial efficacy achieved with herbal ingredients is sometimes not as complete as consumers and manufacturers desire. Moreover, there can be hurdles in developing stable oral care formulations that contain various extracts and oils. Although, in certain oral care formulations containing herbal ingredients, even where the stability problems can be overcome, there can still be issues with antibacterial efficacy. Consequently, it is desirable to provide oral care products containing herbal ingredients having acceptable antibacterial effectiveness and also possess an acceptable level of stability.

Accordingly, there is a need in the space for a product that can stably contain a mixture of herbal ingredients (e.g., extracts and oils) but at the same time also deliver acceptable results with respect to antibacterial activity and overall efficacy.

### BRIEF SUMMARY

Compositions of the disclosure can surprisingly offer the advantage of providing a naturals flavor profile along with a robust antimicrobial protection. Additionally, the compositions disclosure are believed to have acceptable stability profiles which allow for acceptable storage and subsequent consumer use. The formulations of the disclosure are also believed to have acceptable aesthetic qualities (e.g., avoid poor taste) and are generally acceptable for consumers.

In one aspect, the compositions of the disclosure have acceptable viscosity and stability that were not shared by previous formulations that employed, for example, certain extracts and contained a carrageenan gum system. In one aspect, the compositions of the disclosure utilize a gum system comprising xanthan gum and carboxymethyl cellulose which is believed to impart stability to formulations that contain herbal extracts, oils and oleoresins. In one aspect, the incorporation of a gum system comprising carboxymethyl cellulose and xanthan gum (e.g., in a ratio of 1:2 to 1:5 (CMC to xanthan gum)) (e.g., 3.5:1, CMC to xanthan gum) is believed to surprisingly provide stability to formulas that contain ginger and pepper oleoresins. In this aspect, it is believed that these formulas containing ginger and pepper oleoresins also possess acceptable antibacterial efficacy in addition to acceptable formula stability.

In some embodiments, the present invention provides an oral care composition comprising: An oral care composition comprising: a first source of zinc comprising zinc oxide; a second source of zinc comprising zinc citrate; a gum system comprising xanthan gum and carboxymethyl cellulose; a carrier comprising calcium carbonate (e.g., natural calcium carbonate and precipitated calcium carbonate); an herbal extract and essential oil; and at least one oleoresin.

Some embodiments provide a method of treating or preventing a disease or condition of the oral cavity comprising contacting an oral cavity surface of a patient in need thereof with any one of the compositions described herein.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In one aspect the invention is an oral care composition (Composition 1.0) comprising:
a. a first source of zinc comprising zinc oxide;
b. a second source of zinc comprising zinc citrate;
c. a gum system comprising xanthan gum and carboxymethyl cellulose;
d. a carrier comprising calcium carbonate (e.g., natural calcium carbonate and precipitated calcium carbonate);
e. a natural ingredient component comprising:
   at least one herbal extract(s) and
   at least one essential oil; and
f. at least one oleoresin (e.g., ginger and/or pepper).
   For example, the invention contemplates any of the following compositions (unless otherwise indicated, values are given as percentage of the overall weight of the composition).
1.1 Composition 1.0, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).
1.2 Any of the preceding compositions comprising zinc citrate and zinc oxide, wherein the zinc citrate is in an amount of from 0.25 to 1 wt % (e.g., 0.5 wt. %) and zinc oxide may be present in an amount of from 0.75 to 1.25 wt % (e.g., 1.0 wt. %) based on the weight of the oral care composition.
1.3 Any of the preceding compositions, wherein the zinc citrate is in an amount of from 0.3 to 0.8 wt % (e.g., 0.5 wt. %) and zinc oxide may be present in an amount of from 0.8 to 1.2 wt % (e.g., 1.0 wt. %) based on the weight of the oral care composition.
1.4 Any of the preceding compositions, wherein the zinc citrate is in an amount of from 0.4 to 0.6 wt % (e.g., 0.5 wt. %) and zinc oxide may be present in an amount of from 0.9 to 1.1 wt % (e.g., 1.0 wt. %) based on the weight of the oral care composition
1.5 Any of the preceding compositions wherein the zinc citrate is about 0.5 wt % (e.g., zinc citrate trihydrate).
1.6 Any of the preceding compositions wherein the zinc oxide is about 1.0 wt %.
1.7 Any of the preceding compositions where the zinc citrate is about 0.5 wt % and the zinc oxide is about 1.0 wt %.
1.8 Any of the preceding compositions wherein the composition comprises one or more herbal extract(s) and one or more essential oils selected from the group consisting of amla extract, honey extract, almond extract, aloe vera extract, maricha extract, ginger extract, fenugreek, neem seed oil, sesame oil, cinnamon leaf oil, clove oil, thyme oil, eucalyptus oil, eugenol, menthol, babool and camphor.
1.9 Any of the preceding compositions wherein the herbal extracts and essential oils are selected from amla extract, honey extract, neem seed oil, basil oil, aloe vera extract, cinnamon leaf oil, clove oil, and camphor.
1.10 Any of the preceding compositions, wherein any one of the herbal extracts and essential oils can be in an amount from 0.0001%-0.5% by wt.
1.11 Any preceding compositions, wherein the gum system comprises carboxymethyl cellulose (free form or a salt, e.g., sodium salt) and xanthan gum in a weight ratio 1:2 to 1:5, respectively (e.g., about 1:2.5), (e.g., about 1:3), (e.g., about 1:3.5).
1.12 The preceding composition, wherein the ratio of carboxymethylcellulose to xanthan gum is about 3.5:1.
1.13 Any of the preceding compositions, wherein the gum system comprises carboxymethylcellulose from 0.25%-1% by wt. relative to the total composition (e.g., about 0.6% by wt.) (e.g., about 0.7% by wt.) (e.g., about 0.75% by wt.) (e.g., about 0.8% by wt.).
1.14 Any of the preceding compositions, wherein the gum system comprises xanthan gum from 0.05%-0.5% by wt. relative to the total composition (e.g., about 0.1% by wt.) (e.g., about 0.2% by wt.) (e.g., about 0.3% by wt.) (e.g., about 0.4% by wt.).
1.15 Any of the preceding compositions, wherein the gum system comprises carboxymethylcellulose from 0.25%-1% by wt. (e.g., about 0.7% by wt.) and xanthan gum from 0.05%-0.5% by wt. (e.g., about 0.2%) relative to the total composition.
1.16 Any of the preceding compositions, wherein the gum system further comprises one or more selected from: gum arabic, polyethylene glycol (e.g., polyethylene glycol 200, 400, 600 or 800, or a mixture thereof), and combinations thereof.
1.17 Any preceding composition further comprising a silica thickener and/or a silica abrasive.
1.18 Any of the preceding compositions, wherein the oleoresin is selected from the group consisting of: basil, capsicum (paprika), cardamon, celery seed, cinnamon bark, clove bud, fenugreek, fir, balsam, ginger, labdanum, mace, marjoram, nutmeg, parsley, pepper (black or white), pimenta, rosemary, sage, savory, thyme, turmeric, vanilla, bay leaves and combinations thereof.
1.19 The preceding oral care composition, wherein the oleoresin comprises ginger and pepper.
1.20 Any of the preceding compositions, wherein the calcium carbonate comprises precipitated calcium carbonate in an amount from 5-15% by wt. (e.g., refined precipitated calcium carbonate) (e.g., 10 to 13% by weight of the composition) (e.g., 11% precipitated calcium carbonate) (e.g., 12% precipitated calcium carbonate).
1.21 Any of the preceding compositions, wherein the calcium carbonate comprises natural calcium carbonate in an amount from 20-40% by wt. (e.g., 25 to 35% by weight of the composition) (e.g., 30% natural calcium carbonate).
1.22 Any of the preceding compositions, wherein the oral care composition comprises:
a. From 0.5-1.25% by wt. zinc oxide (e.g., about 1.0%)
b. From 0.25-0.75% by wt. zinc citrate (e.g., about 0.5%)
c. From 0.3%-1% sodium monofluorphosphate (e.g., about 0.76%)
d. wherein the herbal extract and essential oil comprise: amla extract, neem seed oil, fennel oil, basil oil, cinnamon leaf oil, honey extract and aloe vera extract;
e. wherein the gum system comprises carboxymethylcellulose from 0.25%-1% by wt. (e.g., about 0.7% by wt.) and xanthan gum from 0.05%-0.5% by wt. (e.g., about 0.2%) relative to the total composition; and
f. wherein the oleoresin comprises pepper (e.g., black pepper) and ginger.
1.23 An oral care composition of any of the preceding compositions comprising:

| Ingredient | Wt. % |
|---|---|
| Humectant (70%) | 23.0 |
| Xanthan gum | 0.2 |
| Carboxymethyl cellulose | 0.7 |
| Sweetener | 0.15 |
| Sodium Monofluorophosphate-USP | 0.76 |
| pH Adjusting Agent | 0.50 |
| Purified Water | q.s. |
| Thickener | 1.75 |
| Refined Natural Calcium Carbonate | 30.0 |
| Precipitated Calcium Carbonate | 12.0 |
| Anionic Surfactant | 2.5 |
| Zinc Oxide | 1.0 |
| Zinc Citrate Trihydrate | 0.5 |
| Desensitizing agent | 0.3 |
| Sodium silicate | 1.00 |
| Amla Extract | 0.10 |
| Honey Extract | 0.0004 |
| Neem Seed Oil | 0.010 |
| Fennel Oil | 0.0004 |
| Basil Oil | 0.0004 |
| Aloe Vera Extract | 0.0004 |
| Cinnamon Leaf Oil | 0.0004 |
| Flavor | 1.80 |
| Colorants | 0.35 |
| Preservative | 0.30 |
| Ginger oleoresin | 0.006 |
| Black pepper oleoresin | 0.006 |
| Total | 100.0 |

1.24 An oral care composition of any of the preceding compositions, wherein the oral care composition is in the form selected from: a dentifrice, toothpaste, transparent paste, gel, mouth rinse, mouthwash, spray, ribbon and chewing gum.

As used throughout, ranges are used as a short hand for describing each and every value that is within the range. Any value within the range can be selected as the terminus of the range.

In addition, all references cited herein are hereby incorporated by reference in their entireties. In the event of a conflict in a definition in the present disclosure and that of a cited reference, the present disclosure controls.

In some embodiments, the compositions further comprise one or more components selected from a fluoride ion source; a tartar control agent; a buffering agent; an antibacterial agent; an abrasive; and a combination of two or more thereof.

As used herein, “oleoresin”, refers to semi-solid extracts composed of resin and essential or fatty oil, obtained by evaporation of the solvents used for their production. Naturally occurring oleoresins are also known as balsams.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

Some embodiments provide compositions wherein at least one of the one or more components is a fluoride ion source selected from: stannous fluoride, sodium fluoride, potassium fluoride, sodium monofluorophosphate, sodium fluorosilicate, ammonium fluorosilicate, amine fluoride, ammonium fluoride, and a combination of two or more thereof.

Other optional additives may be included. Among such optional additives, included are those provided in order to change appearance or aesthetic appeal, and/or to preserve the final product, and/or for taste/cosmetic appeal and/or as therapeutic and prophylactic ingredients for oral health, prevention or treatment of a condition or disorder of hard or soft tissue of the oral cavity, or the prevention or treatment of a physiological disorder or condition.

Some embodiments provide a composition wherein a preservative is present. In some embodiments, the preservative is selected from parabens, potassium sorbate, benzyl alcohol, phenoxyethanol, polyaminopropryl biguanide, caprylic acid, sodium benzoate and cetylpyridinium chloride. In some embodiments, the preservative is present at a concentration of about 0.0001 to about 1%, by weight.

Colorants such as dyes may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-5-{(2-methoxy-5-methyl-4-sulphophenyl)azo}-2-n-aphthalenesulfonic acid, Food Yellow 13, sodium salt of a mixture of the mono and disulphonic acids of quinophtalone or 2-(2-quinolyl) indanedione, FD&C Yellow No. 5 (sodium salt of 4-p-sulfophenylazo-1-p -sulfophenyl-5-hydroxypyrazole-3 carboxylic acid), FD&C Yellow No. 6 (sodium salt of p -sulfophenylazo-B-naphtol-6-monosulfonate), FD&C Green No. 3 (disodium salt of 4-{[4-(N -ethyl-p-sulfobenzylamino)-phenyl]-(4-hydroxy-2-sulfoniumphenyl)-methylene}-[1-(N-ethyl -N-p-sulfobenzyl)-DELTA-3,5-cycl-ohexadienimine], FD&C Blue No. 1 (disodium salt of dibenzyldiethyl-diamino-triphenylcarbinol trisulfonic acid anhydrite), FD&C Blue No. 2 (sodium salt of disulfonic acid of indigotin) and mixtures thereof in various proportions. Typically, colorants if included are present in very small quantities.

Flavor profile is provided by various ingredients which include, but are not limited to, herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract) synthetic flavor oils and flavoring aromatics, and/or oils, oleo resins and extracts derived from herbs plants, leaves, flowers, fruits, honey and so forth, and combinations thereof. Representative herbal extracts include: spearmint oil, cinnamon oil, peppermint oil, clove oil, bay oil, thyme oil, cedar leaf oil, oil of nutmeg, oil of sage, and oil of bitter almonds. Examples of herbal extracts include mints such as peppermint, artificial vanilla, cinnamon derivatives, and various fruit flavors, whether employed individually or in admixture. Generally, any flavoring agent or food additive, such as those described in Chemicals Used in Food Processing, publication 1274 by the National Academy of Sciences, pages 63-258, may be used. Typically, the total amount of herbal extract or essential oil is from about 0.1 to about 5%, by weight; from about 0.2 to about 4% by weight; about 0.5 to about 3% by weight; about 1 to about 2.5% by weight; from 1 to about 3% by weight.

In one aspect, any of Composition 1 et seq comprise herbal extracts and essential oils that include amla extract, honey extract, almond extract, aloe vera extract, maricha extract, ginger extract, fenugreek, neem seed oil, sesame oil, cinnamon leaf oil, clove oil, thyme oil, eucalyptus oil, eugenol, menthol, babool and camphor.

In one aspect, the compositions of the disclosure, e.g., any of Composition 1 et seq., can include a sweetener. Sweeteners include both natural and artificial sweeteners. Suitable sweeteners include water soluble sweetening agents such as monosaccharides, disaccharides and poysaccharides such as xylose, ribose, glucose (dextrose), mannose, galactose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame). In general, the effective amount of sweetener is utilized to provide the level of sweetness desired for a particular composition, will vary with the sweetener selected. This amount will normally be from about 0.001 to about 5%, by weight. In some embodiments, the sweetener is sodium saccharin and is present at a concentration of about 0.01%, by weight.

Whitening agents, material which is effective to effect whitening of a tooth surface to which it is applied, such as hydrogen peroxide and urea peroxide, high cleaning silica, preservatives, silicones, and chlorophyll compounds may be incorporated into the compositions of the present invention. In various embodiments, the compositions of this invention comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof. In some embodiments, the peroxide compound comprises hydrogen peroxide. In some embodiments, the peroxide compound consists essentially of hydrogen peroxide. In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. One or more whitening agents are optionally present in a tooth-whitening effective total amount. In some embodiments the whitening agent is separated from the aqueous carrier. In some embodiments the whitening agent is separated from the aqueous carrier by encapsulation of the whitening agent.

Optionally, an antiplaque (e.g., plaque disrupting) agent may be included. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

Optional desensitizing agents include potassium citrate, potassium chloride, potassium tartrate, potassium bicarbonate, potassium oxalate, potassium nitrate, strontium salts, and mixtures thereof.

Optional additives also include vitamins, and proteins. Vitamins include Vitamins C and D, thiamine, riboflavin, calcium pantothenate, niacin, folic acid, nicotinamide, pyridoxine, cyanocobalamin, para-aminobenzoic acid, bioflavonoids, pantheon, retinyl palmitate, tocopherol acetate, and mixtures thereof. Suitable proteins include milk proteins and enzymes such as peroxide-producing enzymes, amylase, plaque-disrupting agents such as papain, glucoamylase, glucose oxidase, and “next generation” enzymes.”

In some embodiments, the composition has a free water content of greater than about 10%, by weight. In some embodiments, the composition has a free water content of greater than about 11%, by weight. In other embodiments, the composition has a free water content of greater than about 12%, by weight. Yet other embodiments provide compositions wherein the free water content is greater than about 13%, by weight. Still other embodiments provide compositions having a free water content of greater than about 14%, by weight. In some embodiments, the composition has a free water content of greater than about 15%, by weight. While other embodiments provide compositions have a free water content of greater than about 16%, by weight. In some embodiments, the composition has a free water content of about 17%, by weight. In some embodiments, the composition has a free water content of greater than about 17%, by weight. In some embodiments, the composition has a free water content of from about 10% to about 20%, by weight.

Some embodiments provide a method of treating or preventing a disease or condition of the oral cavity comprising contacting an oral cavity surface of a patient in need thereof with any one of the compositions described herein. In other embodiments, the disease or condition of the oral cavity is halitosis. In some embodiments, the present invention provides a method of reducing volatile sulfur compounds in the oral cavity of a subject in need thereof. In further embodiments, the present invention provides a method for increasing the delivery of a metal ion to an oral cavity surface.

In certain embodiments, the compositions described herein can be used, for example, for cavity prevention, whitening, plaque prevention or reduction, gingivitis prevention or reduction, tartar control, breath malodor prevention or reduction, and stain prevention.

The specific composition of the carrier preferably depends on the intended use of the composition. In various embodiments, the carrier is aqueous, comprising from about 5 to about 95%, by weight, water or from about 10 to about 70%, by weight, water. In other embodiments, the carrier is substantially non-aqueous. In a dentifrice carrier, water content can be from about 5 to about 70%, from about 10 to about 50%, or from about 20 to about 40%, by weight.

The carrier may comprise any of a variety of materials, including emulsifiers, thickeners, fillers, and preservatives. In some embodiments, the carrier may include a functional or active material, such as those described above.

In some embodiments, the carrier comprises a humectant, such as glycerin, sorbitol or an alkylene glycol such as polyethylene glycol or propylene glycol. In some embodiments, the carrier comprises a humectant at a level of from about 10 to about 80% by weight, or about 20 to about 60% by weight of the composition. Carrier compositions among those useful herein are disclosed in U.S. Pat. No. 5,695,746 to Garlick, Jr., et al. and U.S. Pat. No. 4,839,157 to Mei-King Ng et al.

### Fluoride Ion Source

The oral care compositions of the disclosure may, e.g., any of Composition 1.0 et seq., can further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The oral care compositions of the disclosure may, e.g., any of Composition 1.0 et seq., comprise anionic surfactants, for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOS0₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OS0₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃o alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., about 1.75% by wt.

In another embodiment, cationic surfactants useful in compositions of the present disclosure, e.g., any of Composition 1.0 et seq, can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be included in compositions of the disclosure, e.g., any of Composition 1.0, et seq., can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

Illustrative amphoteric surfactants that can be used in the compositions of the disclosure, e.g., any of Composition 1.0 et seq., include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

The oral care compositions of the disclosure, e.g., any of Composition 1.0 et seq, may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

In one aspect, the flavoring agent is incorporated in the oral composition at a concentration of 0.01 to 1% by weight.

### pH Adjusting Agents

In some embodiments, the compositions of the present disclosure, e.g., any of Composition 1.0 et seq., contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tripolyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

The oral care compositions of the disclosure, e.g., any of Composition 1.0 et seq., also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt %. The pyrophosphates also contribute to preservation of the compositions by lowering water activity.

Suitable anticalculus agents for compositions of the disclosure (e.g., any of Composition 1.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the invention includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂0₇), calcium pyrophosphate (Ca₂P₂0₇), and sodium phosphate dibasic (Na₂HP0₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP) (Na₅P₃0₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the disclosure, e.g., any of Composition 1.0 et seq., also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly (N-vinyl-2-pyrrolidinone), poly (N-vinylbutyrolactam), poly( 1-vinyl-2-pyrrolidone) and poly [1-(2-oxo-1 pyrrolidinyl) ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, e.g., any of Composition 1.0 et seq., the thickening agents are carboxyvinyl polymers, xanthan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, e.g., any of Composition 1.0 et seq., microcrystalline cellulose (MCC) can be used (e.g., carboxymethyl cellulose with sodium carboxymethyl cellulose) in compositions of the disclosure. An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC. In certain such embodiments, the MCC/sodium carboxymethylcellulose may be present in an amount of from 0.5 to 1.5 weight % based on the total weight of the composition.

### Abrasives

In some aspects, compositions of the disclosure, e.g., any of Composition 1.0 et seq., can comprise an abrasive. Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g., about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(P0₄)₂), hydroxyapatite (Ca₁₀(P0₄)₆(OH)₂), or dicalcium phosphate dihydrate (CaHP0₄·2H₂0, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example, synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

### Amino Acids

In some aspects, the compositions of the disclosure, e.g., any of Compositions 1.0 et seq, can include a basic or neutral amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, the compositions of the disclosure (e.g., any of Compositions 1.0 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the invention (e.g., Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Water

Water is present in the oral compositions of the invention. Water, employed in the preparation of commercial oral compositions should be deionized and free of organic impurities. Water commonly makes up the balance of the compositions and can include 5% to 90%, e.g., 10% to 90%, e.g., 20-75%, e.g., 25-75%, e.g., 30%-75%, e.g., 35%-65%, e.g., 40%-65%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, e.g., any of Composition 1.0, et seq., it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

In one aspect, the present disclosure in its method aspect involves applying to the oral cavity a safe and effective amount of the compositions described herein, e.g., any of Composition 1.0 et seq.

The compositions and methods according to the invention (e.g., Composition 1.0 et seq) can be incorporated into oral compositions for the care of the mouth and teeth such as dentifrices, toothpastes, transparent pastes, gels, mouth rinses, sprays and chewing gum.

Other ingredients which may optionally be included in compositions according to the present invention include hyaluronic acid, green tea, ginger, sea salt, coconut oil, turmeric, white turmeric (white curcumin), grape seed oil, ginseng, monk fruit, vitamin E, basil, chamomile, pomegranate, aloe vera, and charcoal. Any of such ingredients may be present in an amount from 0.01% to 2% by weight of the composition, e.g., 0.01 to 1%, or 0.01 to 0.5%, or 0.01 to 0.1%.

Compositions as described herein can be prepared according to methods readily known to those skilled in the art.

Embodiments of the present invention are further described in the following examples. The examples are merely illustrative and do not in any way limit the scope of the invention as described and claimed.

### EXAMPLES

A series of formulations is prepared and evaluated on a range of physical characteristics, and display unacceptable performance either as initially formulated or upon storage under the stated conditions, as detailed below.

### Example 1

The following is a representative formula of the present disclosure:

TABLE 1
Formula A

| Ingredient | Wt. % |
|---|---|
| Humectant (70%) | 23.0 |
| Xanthan gum | 0.2 |
| Carboxymethyl cellulose | 0.7 |
| Sweetener | 0.15 |
| Sodium Monofluorophosphate-USP | 0.76 |
| pH Adjusting Agent | 0.50 |
| Purified Water | q.s. |
| Thickener | 1.75 |
| Refined Natural Calcium Carbonate | 30.0 |
| Precipitated Calcium Carbonate | 12.0 |
| Anionic Surfactant | 2.5 |
| Zinc Oxide | 1.0 |
| Zinc Citrate Trihydrate | 0.5 |
| Desensitizing agent | 0.3 |
| Sodium silicate | 1.00 |
| Amla Extract | 0.10 |
| Honey Extract | 0.0004 |
| Neem Seed Oil | 0.010 |
| Fennel Oil | 0.0004 |
| Basil Oil | 0.0004 |
| Aloe Vera Extract | 0.0004 |
| Cinnamon Leaf Oil | 0.0004 |
| Flavor | 1.80 |
| Colorants | 0.35 |
| Preservative | 0.30 |
| Ginger oleoresin | 0.006 |
| Black pepper oleoresin | 0.006 |
| Total | 100.0 |

### Example 2

Formulations of the disclosure are tested for antibacterial efficacy using the growth inhibition University of Manchester model. For this efficacy evaluation, the University of Manchester (UoM) anaerobic biofilm growth is used to provide an indication of potential performance of the tested formulas against anaerobic biofilm. The anaerobic model (UoM) is believed to provide a more sensitive indication of potential efficacy of the formula. In this model, saliva collected from 4 healthy volunteers and pooled together is used as an inoculum. Each sample is treated in triplicate twice a day for 8 days. In this study biofilm is recovered after 16 treatments to measure for ATP (RLU) (Promega BactitreGlo Microbial Cell Viability kit) as an end point for viable bacteria. The formulations of the disclosure tested in the assay are as follow:

| | Wt. % | Wt. % | Wt. % | Wt. % |
|---|---|---|---|---|
| Ingredient | Control 1 | Formula B* | Formula C* | Formula D |
| Humectant (70%) | 30 | 23.0 | 23.0 | 30 |
| Xanthan gum | — | 0.2 | 0.2 | — |
| Carboxymethyl cellulose | — | 0.7 | 0.7 | — |
| Carrageenan | 0.825 | | | 0.825 |
| Sweetener | 0.22 | 0.15 | 0.15 | 0.15 |
| Sodium | 0.76 | 0.76 | 0.76 | 0.76 |
| Monofluorophosphate-USP | | | | |
| pH Adjusting Agent | 0.50 | 0.50 | 0.50 | 0.50 |
| Purified Water | q.s. | q.s | q.s. | q.s. |
| Thickener | 1.75 | 1.75 | 1.75 | 2.5 |
| Refined Natural Calcium | 19.0 | 30.0 | 30.0 | 19.0 |
| Carbonate | | | | |
| Precipitated Calcium | 21.5 | 11.0 | 11.0 | 21.5 |
| Carbonate | | | | |
| Anionic Surfactant | 2.5 | 2.5 | 2.5 | 2.4 |
| Zinc Oxide | 1.0 | 1.0 | 1.0 | 1.0 |
| Zinc Citrate Trihydrate | 0.5 | 0.5 | 0.5 | 0.5 |
| Desensitizing agent | 0.3 | 0.3 | 0.3 | 0.3 |
| Sodium silicate | 1.00 | 1.00 | 1.00 | 1.00 |
| Amla Extract | 0.10 | 0.10 | 0.10 | 0.10 |
| Honey Extract | 0.0004 | 0.0004 | 0.0004 | 0.0004 |
| Neem Seed Oil | 0.010 | 0.010 | 0.010 | 0.010 |
| Fennel Oil | 0.0004 | 0.0004 | 0.0004 | 0.0004 |
| Basil Oil | 0.0004 | 0.0004 | 0.0004 | 0.0004 |
| Aloe Vera Extract | 0.0004 | 0.0004 | 0.0004 | 0.0004 |
| Cinnamon Leaf Oil | 0.0004 | 0.0004 | 0.0004 | 0.0004 |
| Flavor | 1.80 | 1.80 | 1.80 | 1.80 |
| Colorants | 0.28 | 0.35 | 0.35 | 0.6 |
| Preservative | 0.30 | 0.30 | 0.30 | 0.30 |
| Ginger oleoresin | — | 0.006 | 0.006 | 0.006 |
| Black pepper oleoresin | — | 0.006 | 0.006 | 0.006 |
| Licorice | | | | 0.25 |
| Total | 100.0 | 100.0 | 100.0 | 100.0 |

*Formula B and Formula C differ with respect to the type of flavor used in each formula.

Results of the assay are listed in Table 2 below:

TABLE 2

| | Log (RLU) | Std. Dev. |
|---|---|---|
| Negative Control | 4.87 | 0.0251 |
| (Commercial Product)* | | |
| Positive Control | 4.08 | 0.0458 |
| (Internal Standard)** | | |
| Control 1 *** | 4.58 | 0.1340 |
| Control 2 *** | 4.57 | 0.0481 |
| Control 3 *** | 4.32 | 0.2397 |
| Formula B | 4.36 | 0.1600 |
| Formula C | 4.47 | 0.2403 |
| Formula D | 4.22 | 0.3300 |

*Commercial market product that does not contain zinc citrate and zinc oxide;
**Internal standard that comprises zinc citrate and zinc oxide;
*** Separate commercial market products believed to contain at least one herbal extract or essential oil.

Formulas B and C demonstrate a trend toward antibacterial improvement over Control 1 and Control 2. Formulas B and C also demonstrate comparable antibacterial efficacy compared to all three commercial market product controls.

As those skilled in the art will appreciate, numerous changes and modifications may be made to the embodiments described herein without departing from the spirit of the invention. It is intended that all such variations fall within the scope of the appended claims.

1. An oral care composition comprising:
a. a first source of zinc comprising zinc oxide;
b. a second source of zinc comprising zinc citrate;
c. a gum system comprising xanthan gum and carboxymethyl cellulose;
d. a carrier comprising calcium carbonate;
e. a natural ingredient component comprising:
at least one herbal extract(s) and
at least one essential oil(s); and
f. at least one oleoresin.

2. The oral care composition of claim 1, wherein the ratio of the amount of zinc oxide to zinc citrate is from 1.5:1 to 4.5:1.

3. The oral care composition of claim 1, wherein the composition comprises zinc citrate and zinc oxide, wherein the zinc citrate is in an amount of from 0.25 to 1 wt % and zinc oxide may be present in an amount of from 0.75 to 1.25 wt % based on the weight of the oral care composition.

4. The oral care composition of claim 1, wherein the composition comprises one or more herbal extract(s) and one or more essential oils selected from the group consisting of amla extract, honey extract, almond extract, aloe vera extract, maricha extract, ginger extract, fenugreek, neem seed oil, sesame oil, cinnamon leaf oil, clove oil, thyme oil, eucalyptus oil, eugenol, menthol, babool, camphor and combinations thereof.

5. The oral care composition of claim 1, wherein the herbal extracts and essential oils are selected from amla extract, honey extract, neem seed oil, basil oil, aloe vera extract, cinnamon leaf oil, clove oil, camphor and combinations thereof.

6. The oral care composition of claim 1, wherein any one of the herbal extracts and essential oils can be in an amount from 0.0001%-0.5% by wt.

7. The oral care composition of claim 1, wherein the gum system comprises carboxymethyl cellulose and xanthan gum in a weight ratio 1:2 to 1:5, respectively.

8. The oral care composition of claim 1, wherein the weight ratio of carboxymethylcellulose to xanthan gum is about 3.5:1.

9. The oral care composition of claim 1, wherein the gum system comprises carboxymethylcellulose from 0.25%-1% by wt. relative to the total composition.

10. The oral care composition of claim 1, wherein the gum system comprises xanthan gum from 0.05%-0.5% by wt. relative to the total composition.

11. The oral care composition of claim 1, wherein the gum system comprises carboxymethylcellulose from 0.25%-1% by wt. and xanthan gum from 0.05%-0.5% by wt., wherein the weights are relative to the total composition.

12. The oral care composition of claim 1, wherein the gum system further comprises one or more selected from: gum arabic, polyethylene glycol and combinations thereof.

13. The oral care composition of claim 1, wherein the composition further comprises a silica thickener and/or a silica abrasive.

14. The oral care composition of claim 1, wherein the oleoresin is selected from the group consisting of: basil, capsicum, cardamon, celery seed, cinnamon bark, clove bud, fenugreek, fir, balsam, ginger, labdanum, mace, marjoram, nutmeg, parsley, pepper, pimenta, rosemary, sage, savory, thyme, turmeric, vanilla, bay leaves and combinations thereof.

15. The oral care composition of claim 1, wherein the oleoresin comprises ginger and pepper.

16. The oral care composition of claim 1, wherein the calcium carbonate comprises precipitated calcium carbonate in an amount from 5%-15% by wt.

17. The oral care composition of claim 1, wherein the calcium carbonate comprises natural calcium carbonate in an amount from 20%-40% by wt.

18. The oral care composition of claim 1, wherein the oral care composition comprises:
a. From 0.5%-1.25% by wt. zinc oxide
b. From 0.25%-0.75% by wt. zinc citrate
c. From 0.3%-1% sodium monofluorphosphate
d. wherein the herbal extract(s) and essential oil(s) comprise: amla extract, neem seed oil, fennel oil, basil oil, cinnamon leaf oil, honey extract and aloe vera extract;
e. wherein the gum system comprises carboxymethylcellulose from 0.25%-1% by wt. and xanthan gum from 0.05%-0.5% by wt., relative to the total composition; and
f. wherein the oleoresin comprises pepper and ginger.

19. A method of treating or preventing a disease or condition of the oral cavity comprising contacting an oral cavity surface of a patient in need thereof with the oral care composition according to claim 1.

20. The method according to claim 19, wherein the disease or condition of the oral cavity is a disease or condition caused by oral bacteria.
